(19) United States
(12) Patent Application Publication
(10) Pub. No.: US 2016/0354302 A1
(43) Pub. Date: Dec. 8, 2016

(54) METHODS INVOLVING SKIN COSMETICS

(71) Applicant: Deborah MITCHELL, Shifnal Shropshire (GB)

(72) Inventor: Deborah MITCHELL, Shifnal Shropshire (GB)

(21) Appl. No.: 15/110,274

(22) PCT Filed: Jan. 7, 2014
(86) PCT No.: PCT/GB2014/050032

§ 371 Date: Jul. 7, 2016

(51) Int. Cl. A61K 8/97; A61K 9/06; A61K 31/425; A61K 31/355; A61Q 19/06; A61K 8/67; A61K 8/49; A61Q 19/08; A61Q 19/10; A61Q 19/00; A61K 9/00; A61K 36/61

(52) U.S. Cl. CPC A61K 8/97; A61K 9/0014; A61K 9/06; A61K 31/425; A61K 31/355; A61K 36/61; A61K 8/678; A61K 8/49; A61K 8/676; A61Q 19/08; A61Q 19/10; A61Q 19/00; A61Q 19/06; A61K 2800/72; A61K 2800/805

(57) ABSTRACT

The invention provides a method for obtaining a composition, the method comprising the steps of: (a) combining an amount of an active agent with a carrier to provide a diluted formulation; (b) taking an amount of diluted formulation and combining it with carrier to provide a more diluted formulation; (c) taking an amount of the more diluted formulation and combining it with carrier to provide a yet more diluted formulation, (d) repeatedly carrying out the step of taking the most diluted version of the formulation and combining an amount of said formulation with carrier, until a final formulation is obtained where the concentration of the active agent is less than 1×10⁻¹⁰ ppm; wherein the steps of (a) to (d), in which active agent is combined with carrier, are carried out over a period of time of an hour or more; and then (e) combining an amount of the final formulation with one or more skin composition ingredients, to provide a skin composition where the concentration of the active agent is less than 1×10⁻¹¹ ppm.

### FIELD OF THE INVENTION

The present invention relates to the field of skin cosmetics, in particular methods for preserving compositions for topical skin application.

### BACKGROUND TO THE INVENTION

Topical creams, ointments and pastes have been used for centuries to help alleviate skin conditions such as burns, infection and damage. Many products are also available to revitalize aging skin or reduce wrinkles.

Compositions for topical skin application can be prone to deterioration over time. This may be due to the fact that one or more of the ingredients of the composition goes off. However, more commonly the deterioration is due to the composition going mouldy. The growth of mould in the composition can in particular occur after long term storage in warm conditions. Mould growth is more likely to occur once the composition is exposed to the air, i.e. after the container within which the composition is housed is opened.

Consumers are likely to throw away compositions for topical skin application if they see that the product has any mould growth. Therefore the product goes to waste. The consumer may also be deterred from purchasing the product again if it has previously gone mouldy.

In addition, many preservatives are known to cause rashes and other allergic or intolerance reactions. For example, parabens are known to cause such problems.

The preservative methylchloroisothiazolinone can cause skin reactions in people.

People may have reactions to some essential oils, e.g. geranium, bergamot, rosemary and/or tea tree oil.

People may also have reactions to bee products, such as propolis, royal jelly, bee venom, and the like.

There is a desire to ensure that compositions intended for application to the human skin are unlikely to cause negative reactions when applied to the skin, such as allergies.

An aim of the invention is therefore to provide a method of obtaining compositions, especially those for topical skin application.

The composition may be one that has an active agent present (e.g. a preservative or other active) where there is a desire to avoid or reduce certain characteristics associated with that agent, e.g. skin reactions or unpleasant odors.

The invention aims to provide the desired effect of the active agent in the compositions (e.g. anti-bacterial or anti-aging or toning or astringent) whilst avoiding or reducing negative reactions such as allergic or intolerance reactions.

Another aim of the invention is to provide a method of preserving compositions, especially those for topical skin application.

The invention aims to provide improved preservation for compositions, whilst avoiding or reducing negative reactions such as allergic or intolerance reactions.

The compositions may in particular be those that are intended to used for cosmetic application, to improve the appearance of the skin, e.g. they may be skin moisturisers, skin toners, anti-aging products, anti-wrinkle products, anti-cellulite products, eye creams, or other skin products for the face or body.

### SUMMARY OF THE INVENTION

According to a first aspect of the present invention, there is provided a method for obtaining a composition, the method comprising the steps of:

(a) combining an amount of an active agent with a carrier to provide a diluted formulation;

(b) taking an amount of diluted formulation and combining it with carrier to provide a more diluted formulation;

(c) taking an amount of the more diluted formulation and combining it with carrier to provide a yet more diluted formulation,

(d) repeatedly carrying out the step of taking the most diluted version of the formulation and combining an amount of said formulation with carrier, until a final formulation is obtained where the concentration of the active agent is less than 1×10⁻¹⁰ ppm;

wherein the steps of (a) to (d), in which active agent is combined with carrier, are carried out over a period of time of an hour or more; and then

(e) combining an amount of the final formulation with one or more skin composition ingredients, to provide a skin composition where the concentration of the active agent is less than 1×10⁻¹¹ ppm.

In this step (e) further carrier may optionally be added. This carrier may be the same carrier material as used in the preceding steps or may be a different carrier material.

Thus the skin composition as obtained comprises one or more carrier, one or more skin composition ingredients, and active agent, where the concentration of the active agent is less than 1×10⁻¹¹ ppm.

One preferred embodiment of the invention uses an active agent which is a preservative agent. However, the invention is not limited to such agents.

In such an embodiment, the method is a method for preserving a composition.

Advantageously, the method of the present invention provides a skin composition that contains the active agent (e.g. preservative agent) at a level so dilute that it does not cause any adverse reaction for the user of the composition. For example, if the user is allergic or intolerant to the agent, no adverse reaction is seen when the skin composition is applied to the user's skin.

However, surprisingly, when the agent is a preservative, the extended contact of the preservative agent with the carrier has been found to impart preservative characteristics thereto, such that the skin composition obtained by the method of the invention is less likely to experience mould growth, with mould growth being prevented or reduced.

Indeed, when the final formulation is combined with one or more skin composition ingredients that are already exhibiting mould growth, the mould in the skin composition ingredients is destroyed or the extent of mould in the skin composition ingredients is reduced.

Equally, if the active agent is not a preservative, the extended contact of the active agent with the carrier may be found to impart the characteristics of that active agent thereto, e.g. anti-aging or toning or astringent.

Active agents such as methylisothiazolinone, tea tree oil and geranium oil are all known as preservative agents but can cause skin reactions.

Bee products are also known as active agents, e.g. with anti-aging properties, but can sometimes cause skin reactions.

According to another aspect of the present invention, there is provided a skin composition obtainable by the method of the invention as described herein.

The composition may be for use in the treatment or prevention of a skin condition selected from: skin dryness, skin ageing, elastosis, laxity (sagging), rhytids (wrinkles), cellulite, skin infection, skin damage, skin burn, pain, muscle tightness and combinations thereof. However, the composition may optionally be used for other conditions.

According to another aspect of the present invention, there is provided a use of the composition according to the invention herein as a topical application on skin. The use may be cosmetic use.

According to another aspect of the present invention, there is provided a cosmetic treatment of the skin, comprising the application of the composition according to the invention herein on the skin.

According to another aspect of the present invention, there is provided a face mask incorporating the composition according to the invention herein.

### DETAILED DESCRIPTION OF THE INVENTION

The method of the present invention makes use of an active agent, which may be a preservative. A key feature of the invention is the low levels of active agent, e.g. preservative, that are present in the end product, meaning that active agents that might otherwise cause a negative reaction can be utilised.

In one embodiment the active agent is a preservative or comprises a preservative.

The preservative may, in one embodiment, be selected from any of the group comprising phenoxyethanol; methylchloroisothiazolinone; salicylic acid; potassium sorbate; DMDM hydantoin; benzyl alcohol; sodium benzoate; formaldahyde; chlorphenism; triclosan; imidazolidinyl urea; diazolidinyl urea; sorbic acid; methylisothiazolinone; sodium dehydroacetate; dehydroacetic acid; quaternium-15; stearalkonium chloride; zinc pyrithione; sodium metabisulfite; 2-bromo-2-nitropropane; chlorhexidine digluconate; polyaminopropyl biguanide; benzalkonium chloride; sodium sulfite; sodium salicylate; citric acid; grapefruit seed extract; neem oil; essential oil; lactic acid; and vitamin E (tocopherol); and combinations thereof.

The preservative may, in one embodiment, be selected from any of the group comprising phenoxyethanol; methylchloroisothiazolinone; grapefruit seed extract; neem oil; essential oil (e.g. tea tree oil and/or geranium oil and/or rosemary oil and/or bergamot oil); and vitamin E (tocopherol); and combinations thereof.

The preservative may, in one embodiment, be selected from any of the group comprising methylchloroisothiazolinone; essential oil (e.g. tea tree oil and/or geranium oil and/or rosemary oil and/or bergamot oil); and vitamin E (tocopherol); and combinations thereof.

The preservative may in one embodiment comprise methylchloroisothiazolinone.

In one embodiment the active agent comprises one or more bee products. Bee products may, for example, comprise honey (e.g. manuka honey), propolis, honey wax, bee pollen, royal jelly, bee venom, or combinations thereof. In one embodiment, the active agent comprises one or more of honey, propolis, bee venom and royal jelly. In one embodiment, the active agent comprises one or more of propolis, royal jelly, and bee venom.

In one embodiment, the composition as made by the method of the invention does not comprise parabens.

In step (d) the method involves repeatedly carrying out the step of taking the most diluted version of the formulation and combining an amount of said formulation with carrier, until a final formulation is obtained where the concentration of the agent is less than 1×10⁻¹¹ ppm. Preferably the step is repeated until a final formulation is obtained where the concentration of the agent is less than 1×10⁻¹¹ ppm, e.g. less than 1×10⁻¹² ppm or less than 1×10⁻¹³ ppm or less than 1×10⁻¹⁴ ppm or less than 1×10⁻¹⁵ ppm.

In the method of the invention the steps of (a) to (d), in which agent is combined with carrier, are carried out over a period of time of an hour or more. Preferably the steps of (a) to (d), in which agent is combined with carrier, are carried out over a period of time of two hours or more, such as three hours or more, or four hours or more, or five hours or more.

In the method of the invention the step (e) involves combining an amount of the final formulation with one or more skin composition ingredients, so as to provide a skin composition where the concentration of the agent in the final formulation is less than 1×10⁻¹¹ ppm. Preferably the concentration of the agent in the final formulation is less than 1×10⁻¹² ppm, e.g. less than 1×10⁻¹³ ppm or less than 1×10⁻¹⁴ ppm or less than 1×10⁻¹⁵ ppm or less than 1×10⁻¹⁶ ppm.

In the invention the step (d) involves repeatedly carrying out a dilution step of taking the most diluted version of the formulation and combining an amount of said formulation with carrier. In one embodiment this involves repeating the dilution step ten or more times, such as 20 or more times, e.g. 30 or more times or 40 or more times. Preferably this involves repeating the dilution step 50 or more times, such as 60 or more times, e.g. 70 or more times or 80 or more times. Most preferably this involves repeating the dilution step 90 or more times.

In one embodiment, step (b) of taking an amount of diluted formulation and combining it with carrier to provide a more diluted formulation involves diluting the formulation by a factor of 10 or more, such as 20 or more or 30 or more or 40 or more; preferably by a factor of 50 or more, e.g. by a factor of about 100 or more. It may be, for example, that an about 10 ml amount of diluted formulation is combined with about 1 litre of carrier.

In one embodiment, step (c) of taking an amount of the more diluted formulation and combining it with carrier to provide a yet more diluted formulation, involves diluting the more diluted formulation by a factor of 10 or more, such as 20 or more or 30 or more or 40 or more; preferably by a factor of 50 or more, e.g. by a factor of about 100 or more. It may be, for example, that an about 10 ml amount of the more diluted formulation is combined with about 1 litre of carrier.

In one embodiment, in step (d) of repeatedly carrying out the dilution step of taking the most diluted version of the formulation and combining an amount of said formulation with carrier, each dilution step involves diluting the formulation by a factor of 10 or more, such as 20 or more or 30 or more or 40 or more; preferably by a factor of 50 or more, e.g. by a factor of about 100 or more. It may be, for example, that in each of the dilution steps an about 10 ml amount of diluted formulation is combined with about 1 litre of carrier.

In one embodiment, the total number of dilutions carried out in the steps (a) to (d), in which preservative agent is combined with carrier, is 15 or more, such as 20 or more, e.g. 30 or more or 40 or more. Preferably the total number of dilutions carried out in the steps (a) to (d) is 50 or more, such as 60 or more, e.g. 70 or more or 80 or more. Most preferably the total number of dilutions carried out in the steps (a) to (d) is 90 or more, e.g. about 95 or more.

The dilutions carried out in the steps (a) to (d) may be carried out using any suitable equipment known for use in preparing cosmetic formulations. The formulation and carrier may, for example, be combined in a clean or sterile container and stirred using automatic or manual mixing equipment.

The composition made by the method of the invention may be provided in any suitable form, for example it may be provided as a liquid, cream, gel, oil or paste, or similar cosmetic base.

In general, the compositions of the present invention are topical compositions that can be provided in a variety of forms, including but not limited to lotions, milks, mousses, serums, sprays, aerosols, foams, sticks, gels, creams and ointments. In one embodiment, the composition is in the form of a spray or gel and in another embodiment the composition is in the form of a lotion, milk or cream.

The composition may be a water-based composition, oil-based composition, or emulsion composition.

Examples of the water-based composition include skin lotions, beauty essences, water-based gels, and the like, while examples of the oil-based composition include cleansing oil and oil-based gels, and the like. Examples of the emulsion composition include creams, skin milks and sunscreen lotions, and the like, where the types of emulsion include oil in water emulsion (o/w), water in oil emulsion (w/o) and multilayer emulsion (e.g. w/o/w, o/w/o).

All % amounts stated in the application are by weight, unless stated otherwise.

The carrier used in the method of the invention may be selected from cosmetically acceptable carriers. A blend of two or more carriers may be used, or a single carrier may be used. As noted above, in step (e) further carrier may optionally be added and this may be the same or different to the carrier used in the preceding steps.

The carrier may be oil or wax based and/or water based.

For example, in one embodiment the carrier may be water based and may comprise de-ionized water, purified water, natural spring water, rose water or the like. Mixtures of more than one of these may also be used. In one embodiment de-ionized or purified water is used.

The water based carrier may be 100% water or it may comprise components other than water. These may be components known for use in cosmetic formulations. They may include, but are not limited to, agents such as water-soluble moisturizing agents, conditioning agents, anti-microbials, humectants (e.g. glycerin) and/or other water-soluble skin care actives.

In another embodiment, the carrier may be oil or wax based. The oil may be natural oil or synthetic oil, but preferably is natural oil such as a vegetable oil or a nut oil. The oil may be liquid or solid. The wax is preferably a natural wax.

Clearly the oil or wax that is chosen must be able to act as a carrier. It must be able to be readily combined with the preservative agent in the steps of the method of the invention. Preferably it is a material that can easily be blended at room temperature; thus it may be a liquid at room temperature or a solid that is stirrable at room temperature.

Combinations of one or more oils and/or one or more waxes may be used.

Liquid oils that can be mentioned include avocado oil, Camellia oil, turtle bean oil, macadamia nut oil, corn oil, mink oil, olive oil, Canoga oil, egg yolk oil, sesame seed oil, Persic oil, wheatgerm oil, Camellia sasanqua oil, castor oil, linseed oil, safflower oil, sunflower oil, grapeseed oil, apricot oil, shea oil, sweet almond oil, cotton oil, evening primrose oil, palm oil, perilla oil, hazelnut oil, soybean oil, peanut oil, tea seed oil, kaya oil, rice bran oil, rapeseed oil, alfalfa oil, Chinese tung tree wood oil, Japanese tung tree wood oil, jojoba oil, germ oil, poppyseed oil, pumpkin oil, blackcurrant oil, millet oil, barley oil, quinoa oil, rye oil, candlenut oil, passionflower oil, musk rose oil, triglycerine, glyceryl trioctanoate, and glyceryl triisopalmitate.

Solid oils/fats that can be mentioned include cocoa butter, coconut butter, horse fat, hardened coconut oil, palm oil, beef tallow, mutton tallow, hardened beef tallow, palm kernel oil, lard, Japan wax kernel oil, hardened oil, Japan wax, shea butter, and hardened castor oil;

Waxes that can be mentioned include beeswax, candelilla wax, carnauba wax, lanolin, lanolin acetate, liquid lanolin, sugar cane wax, fatty acid isopropyl lanolin, hexyl laurate, reduced lanolin, jojoba wax, hard lanolin, polyoxyethylene (hereinafter referred to as POE), lanolin alcohol ether, POE lanolin alcohol acetate, lanolin fatty acid polyethylene glycol, and POE hydrogenated lanolin alcohol ether. In one embodiment the carrier is not lanolin based.

Ester oils that can be mentioned include isopropyl myristate, cetyl octoate, octyldodecil myristate, isopropyl palmitate, butyl stearate, hexyl laurate, myristyl myristate, decyloleate, hexyldecyl dimethyl octoate, cetyl lactate, myristyl lactate, lanolin acetate, isocetyl stearate, isocetyl iso-stearate, 12-hydroxy cholesteryl stearate, di-2-ethylhexylic acid ethyleneglycol, dipentaerythritol fatty acid ester, N-alkylglycol monoisostearate, neopentylglycol dicaprate, diisostearyl malate, glyceryl di-2-heptyl undecanate, tri-methylol propane tri-2-ethylhexyl acid, tri-methylol propane triisostearate, pentaerythritol tetra-2-ethylhexyl acid, glyceryl tri-2-ethyl-hexanoate, tri-methylol propane triisostearate, cetyl-2-ethylexanoate, 2-ethylhexyl-palmitate, glycerine trimyristate, glyceride tri-2-heptyl undecatoic acid, methyl ester of castor oil fatty acid, oleate oil, acetoglyceride, palmitate-2-heptyl undecyl, diisopropyl adipate, N-lauroyl-L-glutamic acid-2-octyldodecil ester, di-2-heptylundecyl adipate, di-2-ethylhexyl sebacate, myristate-2-hexyldecyl, palmitate-2-hexyldecyl, adipate-2-hexyldecyl, diisopropyl sebacate, and succinate-2-ethylhexyl.

Higher fatty acids that can be mentioned include lauric acid, myristic acid, palmitic acid, stearic acid, behenic acid, oleic acid, 12-hydroxy-stearic acid, undecylenic acid, lanolin fatty acid, isostearic acid, linolic acid, linolenic acid, and eicosapentaenoic acid.

Higher alcohols of straight/branched chain that can be mentioned include lauryl alcohol, cetyl alcohol, stearyl alcohol, behenyl alcohol, myristyl alcohol, oleyl alcohol, cetostearyl alcohol, monostearyl glycerine ether (batyl alcohol), 2-decyltetradecinol, lanolin alcohol, cholesterol, phytosterol, hexyldodecanol, isostearyl alcohol, octyldodecanol.

It may, for example, be that the carrier is oil or wax based and is selected from shea butter, cocoa butter, jojoba oil, olive oil, almond oil, macadamia nut oil, wheat germ oil, evening primrose oil and the like.

The composition may be an oil in water emulsion (o/w) or a water in oil emulsion (w/o).

In one embodiment, in the composition as obtained by the method of the invention the carrier is present in an amount of from 5 to 99.9%, such as from 10 to 99.5%, or from 12 to 99% or from 15 to 98% or from 20 to 97% or from 25 to 95%. It may be that the carrier is present in an amount of from 25 to 90%, such as from 30 to 85% or from 32 to 80% or from 35 to 75%.

In addition to the carrier, the composition will comprise one or more other skin composition ingredients. In this regard, the composition as obtained by the method of the invention may contain from about 0.5 to about 95% by weight of the composition, of such skin composition ingredients; e.g. from about 1% to about 90%, or from about 2% to about 85%, or from about 5% to about 80%, or from about 7% to about 75% or from about 8% to about 70%, by weight of the composition, of such skin composition ingredients.

In one embodiment, the composition as obtained by the method of the invention may contain from about 10 to about 75% by weight of the composition, of such skin composition ingredients; e.g. from about 12% to about 70%, or from about 15% to about 65%, or from about 20% to about 60%, or from about 25% to about 55% or from about 25% to about 50%, by weight of the composition, of such skin composition ingredients.

In another embodiment, the composition as obtained by the method of the invention may contain from about 1 to about 50% by weight of the composition, of such skin composition ingredients; e.g. from about 1% to about 40%, or from about 2% to about 35%, or from about 3% to about 30%, or from about 4% to about 25% or from about 5% to about 20%, by weight of the composition, of such skin composition ingredients.

The Personal Care Product Council's International Cosmetic Ingredient Dictionary and Handbook, Thirteenth Edition, and the CTFA Cosmetic Ingredient Handbook, Second Edition (1992) each describe a wide variety of non-limiting cosmetic and pharmaceutical ingredients commonly used in the skin care industry, which are suitable optional components for use in the compositions of the present invention. Examples of these ingredient classes include: abrasives, absorbents, aesthetic components such as fragrances, pigments, colorings/colorants, plant extracts including essential oils, anti-caking agents, antifoaming agents, antimicrobials, binders, biological additives, buffering agents, bulking agents, chelating agents, colorants, cosmetic astringents, cosmetic biocides, denaturants, drug astringents, emollients, external analgesics, film formers or materials, opacifying agents, pH adjusters, propellants, reducing agents, sequestrants, skin cooling agents, skin protectants, thickeners/viscosity modifiers, vitamins, and combinations thereof.

In one embodiment the composition may comprise bee products. Bee products may, for example, comprise honey (e.g. manuka honey), propolis, honey wax, bee pollen, royal jelly, bee venom, or combinations thereof. In one embodiment, one or more of honey, propolis, bee venom and royal jelly is present in the composition.

In one embodiment, the composition comprises one or more plant extract. The plant extract may be selected from essential oils, extracts from leaves, extracts from stems, extracts from petals, extracts from seeds, extracts from roots and extracts from pollen.

In one embodiment, the composition comprises one or more essential oil.

The essential oil may be selected from basil oil, bay oil, bergamot oil, black pepper oil, cedarwood oil, chamomile oil, cinnamon oil, citronella oil, clary sage oil, eucalyptus oil, fenugreek oil, frankincense oil, geranium oil, ginger oil, grapefruit oil, jasmine oil, lavender oil, lemon oil, lemongrass oil, lime oil, mandarin oil, marjoram oil, melissa oil, myrrh oil, neem oil, neroli oil, orange oil, patchouli oil, peppermint oil, pine oil, rose oil, rosehip oil, rosemary oil, rosewood oil, sage oil, sandalwood oil, sassafras oil, spearmint oil, star anise oil, tangerine oil, tarragon oil, tea tree oil, thyme oil, ylang-ylang oil, and combinations thereof.

In one embodiment, the essential oil is selected from bergamot oil, cedarwood oil, chamomile oil, eucalyptus oil, fenugreek oil, frankincense oil, geranium oil, jasmine oil, lavender oil, lemon oil, marjoram oil, melissa oil, myrrh oil, neem oil, neroli oil, patchouli oil, rose oil, rosehip oil, rosemary oil, sage oil, sandalwood oil, tea tree oil, thyme oil, ylang-ylang oil, and combinations thereof.

In one embodiment, the essential oil is selected from eucalyptus oil, geranium oil, lavender oil, rose oil, rosemary oil, tea tree oil, and combinations thereof.

In one embodiment, the composition comprises one or more plant extract that is not an essential oil. It may be one or more extract obtained from plant leaves, stems, petals, seeds, roots and/or pollen. For example, it may be one or more extract obtained from plant leaves, stems, and/or roots.

In one embodiment, the plant extract is obtained from a plant selected from: aloe vera, basil, birch, burdock, comfrey, chamomile (including German chamomile), calendula, dandelion, echinacea, elderflower, euphrasia (eyebright), green tea, fennel, horsetail, hyssop, lady's mantle, lavender, lemon balm, lime flower, linden, liquorice, marshmallow, nettle, Oregon grape, plantain, pomegranate, rose, rosemary, sage, St. John's wort, yarrow and witch hazel. The extract may be obtained from the plant's leaves, stems, petals, seeds, roots and/or pollen. For example, it may be obtained from the plant's leaves, stems, and/or roots.

In one embodiment, the plant extract is obtained from a plant selected from: aloe vera, comfrey, chamomile (including German chamomile), calendula, echinacea, fennel, green tea, horsetail, hyssop, liquorice, marshmallow, nettle, Oregon grape, pomegranate, and witch hazel. The extract may be obtained from the plant's leaves, stems, petals, seeds, roots and/or pollen. For example, it may be obtained from the plant's leaves, stems, and/or roots.

In one embodiment, the plant extract is obtained from a plant selected from: aloe vera, comfrey, chamomile (including German chamomile), calendula, echinacea, fennel, horsetail and marshmallow. The extract may be obtained from the plant's leaves, stems, petals, seeds, roots and/or pollen. For example, it may be obtained from the plant's leaves, stems, and/or roots.

In one embodiment, the plant extract is obtained from calendula. In particular it may be calendula absolute. The inclusion of calendula stimulates a stronger healing effect. In particular this may be useful for people with sensitive skins, as the desired effect can be achieved with a relatively low concentration of queen bee venom.

In one embodiment, the composition comprises one or more antioxidant.

The antioxidant may be selected from ascorbic acid and derivatives thereof, erythorbic acid and derivatives thereof, and tocopherols (vitamin E forms) and derivatives thereof.

Examples of ascorbic acid or derivatives thereof include ascorbic acid, sodium ascorbate, potassium ascorbate, calcium ascorbate, L-ascorbic acid phosphate ester, magnesium ascorbyl phosphate, sodium ascorbyl phosphate, ascorbyl sulfate, sodium ascorbyl 2 phosphate salt and ascorbyl-2-glucoside, and the like.

Examples of erythorbic acid or derivatives thereof include erythorbic acid or derivative thereof, such as erythorbic acid, sodium erythorbate, potassium erythorbate, calcium erythorbate, erythorbic acid phosphate, erythorbic acid sulfate and the like.

In general, suitable tocopherols include naturally occurring vitamin E, synthetic vitamin E, enantiomerically pure forms of vitamin E (e.g. (+)-alpha-tocopherol), vitamin E derivatives such as acetates, succinates, linoleate, more water-soluble forms of vitamin E such as tocophereth-5, tocophereth-10, tocophereth-12, tocophereth-18, tocophereth-50, D-alpha-tocopherol polyethylene glycol 1000-succinates. Specifica examples of tocopherol or derivatives thereof include α-tocopherol, β-tocopherol, γ-tocopherol, δ-tocopherol, acetic acid-α-tocopherol, nicotinic acid-α-tocopherol, linoleic acid-αtocopherol, succinic acid-α-tocopherol, as well as α-tocotrienol, β-tocotrienol, γ-tocotrienol, and δ-tocotrienol. Particular preference is given to naturally occurring vitamin E.

Other antioxidants that can be mentioned include flavonoids (catechin, anthocyanin, flavone, isoflavone, flavan, flavanone and rutin), phenolic acids (chlorogenic acid, ellagic acid, gallic acid and propyl gallate), lignans, curcumins and coumarins.

These compounds are contained in large amounts in extracts of certain natural substances, so they can be used in the form of extracts. Examples of extracts that can be used include licorice extract, cucumber extract, Millettia dielsiana extract, Gentiana lutea (Japanese gentian) extract, geranium thunbergii extract, cholesterol or derivative thereof, Chinese hawthorn extract, paeoniae radix extract, ginkgo extract, Scutellaria baicalensis extract, carrot extract, Turkestan rose (Ramanas rose) extract, Cassia mimosoides L. extract, tormentil extract, parsley extract, tree peony (peony root bark) extract, flowering quince (chaenomeles) extract, melissa extract, Alnus firma (cornflower) extract, strawberry begonia extract, rosemary extract, lettuce extract, tea extract (oolong tea, red tea, green tea, etc.), microbial fermentation metabolic products and Siraitia grosvenorii extract.

The composition may optionally comprise one or more colouring. In one embodiment, the composition does not contain any added colours and takes its colour from the other ingredients present. In another embodiment, the composition includes natural colorants or pigments.

The composition may comprise one or more perfume. It may be that agents included for other properties, such as essential oils or rose water, also provide a perfuming effect.

In one embodiment, the perfume may be a natural perfume derived from animals, plants or minerals. Examples include rose extract, chamomile extract, green tea aromatic agent, lavender oil, geranium oil, jasmine oil, bergamot oil, musk oil, ylang ylang oil, limonene, linalol, citral, and rose oxide.

When the composition is oil-based, it may include oil-soluble components. Some oil-soluble components that can be mentioned include vitamin E agents, coenzyme Q agents and omega 3 oils (e.g. EPA, DHA, linoleic acid and other oils).

The composition constituents may be natural, non-synthetic, ingredients. It is understood that natural refers to ingredients that exist in or caused by nature and are not made or caused by humankind. Isolated, purified and/or concentrated forms of a natural ingredient may also be considered natural and non-synthetic.

According to another aspect of the present invention, there is provided a skin composition obtainable by the method of the invention as described herein.

The composition may be suitable for use in the treatment or prevention of a skin condition selected from: skin dryness, skin ageing, elastosis, laxity (sagging), rhytids (wrinkles), cellulite, skin infection, skin damage, skin burn, pain, muscle tightness and combinations thereof. However, the composition may optionally be used for other conditions.

According to another aspect of the present invention, there is provided a use of the composition according to the invention herein as a topical application on skin. The use may be cosmetic use.

According to another aspect of the present invention, there is provided a cosmetic treatment of the skin, comprising the application of the composition according to the invention herein on the skin.

The treatment may be to treat or prevent a skin condition selected from: skin dryness, skin ageing, elastosis, laxity (sagging), rhytids (wrinkles), cellulite, skin infection, skin damage, skin burn, pain, muscle tightness and combinations thereof. However, the composition may optionally be used for other conditions.

According to another aspect of the present invention, there is provided a face mask incorporating the composition according to the invention herein.

The face mask may comprise a cream, lotion or paste.

The face mask may be provided in the form of a composition that is to be applied directly to the skin to form a mask. The face mask may alternatively be provided in the form of a wrap impregnated or soaked in the composition, wherein the wrap is to be applied onto the skin.

The skilled person will understand that optional features of one embodiment or aspect of the invention may be applicable, where appropriate, to other embodiments or aspects of the invention.

### Specific Example of the Invention

Embodiments of the invention will now be described in more detail, by way of example only.

A preservative agent comprising 2 ml methylchloroisothiazolinone, 6 ml vitamin E (tocopherol) and 2 ml tea tree oil was provided.

This 10 ml amount of preservative agent was combined in a container with 1 litre of water (de-ionized/purified) and stirred to provide a diluted formulation.

A 10 ml sample of the diluted formulation was taken. This was combined in a container with 1 litre of water (de-ionized/purified) and stirred to provide a more diluted formulation.

A 10 ml sample of the more diluted formulation was then taken. This was combined in a container with 1 litre of water (de-ionized/purified) and stirred to provide a yet more diluted formulation.

This process of taking 10 ml samples of the most diluted formulation and further diluting, by combining in a container with 1 litre of water (de-ionized/purified) and stirring, was repeated until a total of 95 dilution steps had been carried out.

The thus-diluted formulation was then added during the production of a cosmetic skin soothing cream, containing tea tree oil, with the diluted formulation being included at a 10 wt % level.

The cosmetic product had an improved shelf life, as compared to an equivalent product containing tea tree oil that did not have the diluted formulation added.

Thus there was a preservative effect over and above that which could be attributed to the tea tree oil in the cosmetic skin soothing cream.

Additionally, when the cosmetic skin soothing cream was used by people with an allergy or sensitivity to methylchloroisothiazolinone, those people did not show any reaction on their skin to the product.

In a second test, the thus-diluted formulation was added to a cosmetic cleansing lotion containing eyebright extract and vitamin C that had been in the boot of a car for six weeks, and which had been exposed to warm day-time conditions and cool and damp night-time conditions, and which had therefore gone mouldy. Mould was clearly visible when inspecting the product. The diluted formulation was added at a level of about 10 wt % to the lotion and the combined material was stirred. It was then left and re-inspected after a week, at which time there were no visible signs of mould remaining at all.

Additionally, when it was used by people with an allergy or sensitivity to methylchloroisothiazolinone, those people did not show any reaction on their skin to the product.

1. A method for obtaining a composition, the method comprising the steps of:
(a) combining an amount of an active agent with a carrier to provide a diluted formulation;
(b) taking an amount of diluted formulation and combining it with carrier to provide a more diluted formulation;
(c) taking an amount of the more diluted formulation and combining it with carrier to provide a yet more diluted formulation,
(d) repeatedly carrying out the step of taking the most diluted version of the formulation and combining an amount of said formulation with carrier, until a final formulation is obtained where the concentration of the active agent is less than 1×10⁻¹⁰ ppm;
wherein the steps of (a) to (d), in which active agent is combined with carrier, are carried out over a period of time of an hour or more; and then
(e) combining an amount of the final formulation with one or more skin composition ingredients, to provide a skin composition where the concentration of the active agent is less than 1×10⁻¹¹ ppm.

2. The method of claim 1 wherein step (e) further comprises a carrier.

3. The method of claim 1, wherein in step (d) the dilution step is repeated until a final formulation is obtained where the concentration of the agent is:
(a) less than 1×10⁻¹¹ ppm,
(b) less than 1×10⁻¹² ppm,
(c) less than 1×10⁻¹³ ppm,
(d) less than 1×10⁻¹⁴ ppm, or
(e) less than 1×10⁻¹⁵ ppm.

4. The method of claim 1 wherein the steps of (a) to (d), in which agent is combined with carrier, are carried out over a period of time of:
(a) two hours or more,
(b) three hours or more,
(c) four hours or more, or
(d) five hours or more.

5. The method of claim 1 wherein step (e) provides a skin composition where the concentration of the agent in the final formulation is:
(a) less than 1×10⁻¹² ppm,
(b) less than 1×10⁻¹³ ppm,
(c) less than 1×10⁻¹⁴ ppm,
(d) less than 1×10⁻¹⁵ ppm, or
(e) less than 1×10⁻¹⁶ ppm.

6. The method of claim 1 wherein step (d) involves repeating the dilution step:
(a) ten or more times,
(b) 20 or more times,
(c) 50 or more times,
(d) 70 or more times, or
(e) 90 or more times.

7. The method of claim 1 wherein step (b) involves diluting the formulation by a factor of:
(a) 10 or more,
(b) 20 or more,
(c) 50 or more,
(d) 80 or more, or
(e) 100 or more.

8. The method of claim 1 wherein step (c) involves diluting the formulation by a factor of:
(a) 10 or more,
(b) 20 or more,
(c) 50 or more,
(d) 80 or more, or
(e) 100 or more.

9. The method of claim 1 wherein in step (d) each dilution step involves diluting the formulation by a factor of:
(a) 10 or more,
(b) 20 or more,
(c) 50 or more,
(d) 80 or more, or
(e) 100 or more.

10. The method of claim 1 wherein the total number of dilutions carried out in the steps (a) to (d), is
(a) 15 or more,
(b) 30 or more,
(c) 60 or more,
(d) 80 or more, or
(e) 95 or more.

11. The method of claim 1 wherein the active agent which is a preservative agent or comprises a preservative agent.

12. The method of claim 11, wherein the preservative agent is phenoxyethanol; methylchloroisothiazolinone; salicylic acid; potassium sorbate; DMDM hydantoin; benzyl alcohol; sodium benzoate; formaldahyde; chlorphenism; triclosan; imidazolidinyl urea; diazolidinyl urea; sorbic acid; methylisothiazolinone; sodium dehydroacetate; dehydroacetic acid; quaternium-15; stearalkonium chloride; zinc pyrithione; sodium metabisulfite; 2-bromo-2-nitropropane; chlorhexidine digluconate;
polyaminopropyl biguanide; benzalkonium chloride; sodium sulfite; sodium salicylate; citric acid; grapefruit seed extract; neem oil; essential oil; lactic acid; vitamin E; or combinations thereof.

13. The method of claim 11, wherein the preservative agent is phenoxyethanol; methylchloroisothiazolinone; grapefruit seed extract; neem oil; essential oil; vitamin E; or combinations thereof.

14. The method of claim 13, wherein the preservative agent is methylchloroisothiazolinone; tea tree oil; geranium oil; rosemary oil; bergamot oil; vitamin E; or combinations thereof.

15. The method of claim 1, wherein the active agent comprises one or more bee products.

16. The method of claim 15, wherein the bee product is honey, propolis, honey wax, bee pollen, royal jelly, bee venom, or combinations thereof.

17. The method of claim 15, wherein the bee product is propolis, royal jelly, bee venom, or combinations thereof.

18. A skin composition obtainable by a method comprising the steps of:
(a) combining an amount of an active agent with a carrier to provide a diluted formulation;
(b) taking an amount of diluted formulation and combining it with carrier to provide a more diluted formulation;
(c) taking an amount of the more diluted formulation and combining it with carrier to provide a yet more diluted formulation,
(d) repeatedly carrying out the step of taking the most diluted version of the formulation and combining an amount of said formulation with carrier, until a final formulation is obtained where the concentration of the active agent is less than 1×10⁻¹⁰ ppm;
wherein the steps of (a) to (d), in which active agent is combined with carrier, are carried out over a period of time of an hour or more and then
(e) combining an amount of the final formulation with one or more skin composition ingredients, to provide a skin composition where the concentration of the active agent is less than 1×10⁻¹¹ ppm.

19-22. (canceled)

23. A method of treatment of the skin, comprising the application of a composition according to claim 18 on the skin.

24. The method according to claim 23, wherein the method of treatment comprises treatment or prevention of a skin condition, the skin condition being skin dryness, skin ageing, elastosis, laxity, rhytids, cellulite, skin infection, skin damage, skin burn, pain, muscle tightness, or combinations thereof.
